(19) United States
(12) Patent Application Publication
(10) Pub. No.: US 2011/0212480 A1
(43) Pub. Date: Sep. 1, 2011

(54) USE OF PRIMARY HUMAN CARDIOMYOCYTES

(76) Inventors: Kyle Louis Kolaja, Montclair, NJ (US); Preeti Dhawan, San Jose, CA (US); Hirdesh Uppal, San Ramon, CA (US)

(21) Appl. No.: 12/713,985

(22) Filed: Feb. 26, 2010

(51) Int. Cl. C12Q 1/02

(52) U.S. Cl. 435/29

(57) ABSTRACT

The present invention provides for the use of primary human cardiomyocytes for the testing of agents that have cardiotoxic effects and other modulatory effects on the heart.

### BACKGROUND OF THE INVENTION

The heart is an adaptive organ for pumping blood, responding to changing needs by modifying contractile strength and beating rate. The cardiac myocyte is the principal cell in the heart; it coordinates contraction and has the capability to sense a large number of hormonal, neural, electrical and mechanical inputs through a variety of cell surface and nuclear receptors. Myocytes are also targets of an extraordinary number of physiological and pharmacological agents, because of the critical need to regulate contraction strength and heart rate, and their importance in several cardiovascular diseases.

Primary cells isolated from intact heart have been an important model for study because there are no cell lines that maintain the unique rod shaped morphology and complement of proteins necessary for cardiac function. In serum-free culture, adult cardiac myocytes from guinea pigs, rats, mouse and rabbits are usually quiescent and retain their viability and unique rod-shaped morphology for at least a few days. These cells maintain highly organized membrane and myofibrillar structures that support contractions induced by electrical or pharmacological stimulation, and are amenable to viral-mediated expression of exogenous proteins. But similarly successful culture of human cardiac myocytes has been more challenging and not possible, perhaps because of difficulties in enzymatic isolation of healthy myocytes and unique variables for relatively long-term culture. As a consequence, less is known about human cardiac myocyte physiology.

An understanding of cardiotoxicity and of the difficulties in predicting cardiotoxic potential requires insight into the molecular basis of the cardiac function. The understanding of molecular mechanisms of cardiotoxicity has shown that a multitude of extra cellular factors, intracellular factors, transcriptional events and signaling pathways are involved. Thus a large number of players have been shown to be key determinants in the orchestration of a multitude of these pathways to maintain normal cardiac function. Moreover, if dysregulated or inhibited, these extra cellular factors, intracellular factors, transcriptional events and signaling pathways cause the toxicities observed in adverse cardiovascular events. The development of targeted therapies, inhibitors, and drugs has shown some significant liabilities with regards to cardiotoxicity especially in the area of cancer therapy.

Recently, progress has been made in determining basic mechanisms underlying the cardiotoxicity of drugs. There are two key features to clarify for each drug, small molecule compound, ligand, or protein/biotherapuetic that show cardiotoxicity. First, determining the mechanisms of toxicity requires the identification of the specific target responsible for cardiotoxicity. The identification of targets mediating cardiotoxicity can also help to guide future drug development, because some of these molecules or proteins are likely to be ‘bystander’ targets that have no role in the disease indication that a given drug is being developed for and there is therefore no need for the drug to inhibit them. Second, there is a requirement for delineating the mechanisms of toxicity so that the signaling pathways that transduce the toxicity are identified. In some instances, the pathway that leads to cardiomyocyte dysfunction or death will not be the same as the pathway that is crucial for drug action. Therefore, strategies could be developed to block the drug-induced pathways that lead to toxicity but to leave the drug's therapeutic pathways intact.

The development of drugs that inhibit the activity of certain tyrosine kinases for cancer therapy have been associated with toxicity to the heart (Force et al., Drug Discovery Today (2008) 13(17/18), 778-784; Will et al., Toxicological Scineces (2008) 106(1), 153-161). The development of kinase inhibitors (KIs) creates many opportunities for toxicity, not only as a result of the inhibition of desired targets but, probably much more importantly, due to the inhibition of off-target kinases. Cardiotoxicity of a targeted agent was first reported for trastuzumab, the monoclonal antibody that targets the ERBB2 receptor and adverse cardiac effects have also been reported after treatment of patients with imatinib, and are mentioned in the prescribing information for dasatinib (Sprycel), sunitinib (Sutent), sorafenib (Nexavar) and bevacizumab (Avastin). Cardiotoxicity is not associated with all kinase inhibitors because it is not observed with certain other KIs, such as those that target the epidermal growth factor receptor. Therefore, cardiotoxicity needs to be determined for each agent on a case-by-case basis.

### SUMMARY OF THE INVENTION

The present invention provides a method of screening an agent for a modulatory effect on primary human cardiomyocytes comprising culturing said primary human cardiomyocytes, plating said primary human cardiomyocytes on multi-well plates, contacting said primary human cardiomyoctyes with the agent; examining said primary human cardiomyocytes for the modulatory effect resulting from said agent.

In one embodiment of the invention, the screening is performed on primary human cardiomyocytes that are cultured for at least three passages (P=3) in growth medium comprising of Medium 199 or DMEM, 2-10% bovine or fetal calf serum, on plates coated with collagen I.

### DETAILED DESCRIPTION OF THE INVENTION

All publications cited in this disclosure are incorporated herein by reference in their entirety.

### DEFINITIONS

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

The term “primary human cardiomyocyte” refers to human adult cardiomyocytes derived from dissociated human heart tissue (and not from embryonic or pluripotent stem cells) which are capable of undergoing multiple passages in culture.

The term “cardiotoxicity” as used herein refers to compounds that cause direct or indirect injury to cardiomyocytes and the myocardium and that may manifest in certain clinical symptoms which may include: congestive heart failure, ischemia, hypotension, hypertension, arrhythmias (e.g. bradycardia), edema, QT prolongation and conduction disorders, and thromboembolism.

The term “test compound” refers to a substance which is to be tested for cardiotoxicity. The test compound can be a candidate drug or lead compound, a chemical intermediate, environmental pollutant, a mixture of compounds, and the like.

The term “biochemical assays” as used herein refers to in vitro assays performed to evaluate whether compounds may exhibit toxicity to cardiomyocytes. A number of such assays in which primary human cardiomyocytes have been treated with test compounds are described in the concurrently filed U.S. Provisional Patent Application by Bitter et al. entitled, “Assays To Predict Cardiotoxicity”, U.S. Ser. No. 61/______, filed on Feb. 23, 2009 (Attorney Docket No. R0474A-PRO), which is incorporated herein by reference in its entirety.

All patents and publications identified herein are incorporated herein by reference in their entirety.

### General Method

Cardiac myocytes have a complex network of signals that regulates their essential role in the rhythmic pumping of the heart. This network is an appealing model system in which to study the basic principles underlying cellular signaling mechanisms. We have made progress in this effort through the establishment of standardized myocyte isolation and culture procedures and characterization of important signaling responses.

Primary cells isolated from intact heart have been an important model for study because there are no cell lines that maintain the unique rod shaped morphology and complement of proteins necessary for cardiac function. In serum-free culture, adult cardiac myocytes from guinea pigs, rats, mouse and rabbits are usually quiescent and retain their viability and unique rod-shaped morphology for at least a few days. These cells maintain highly organized membrane and myofibrillar structures that support contractions induced by electrical or pharmacological stimulation, and are amenable to viral-mediated expression of exogenous proteins. But similarly successful culture of human cardiac myocytes has been more challenging and not possible, perhaps because of difficulties in enzymatic isolation of healthy myocytes and unique variables for relatively long-term culture. As a consequence, less is known about human cardiac myocyte physiology.

Our initial goal was therefore to establish procedures for isolation of healthy rod-shaped human myocytes that could be maintained in culture for 24 hours, thereby providing a population of cells suitable for studies on short-term responses to ligands, small molecule compounds, chemicals, drugs, and protein biotherpaeutics. In addition, procedures for maintaining myocytes for 72 hours or longer were needed to allow manipulation of gene expression in culture using antisense oligonucleotides and RNA interference. These rod-shaped myocytes must retain excitation-contraction coupling mechanisms and responses to receptor activation, particularly protein phosphorylation events that affect contractility and hypertrophy.

We have established a standardized procedure for the isolation of ventricular cardiac myocytes from human heart tissue. We have focused our initial development of isolation protocols and assessments of biochemical, anatomical, physiological, imaging-based, pathological and pathway responses by protein phosphorylation with phosphospecific antibodies. The excitation-contraction coupling responses have been measured by measuring changes in cytosolic Ca2+ and contraction with electrical stimulation. Merging these efforts resulted in a reliable and reproducible method that yields cells of quantity and quality sufficient for human cardiac studies in culture. We obtain a large number of rod-shaped myocytes from heart tissue, of which 80% are rod-shaped when freshly isolated. A high percentage of cells maintain a functional, rod-shaped morphology after recovery over 24 hours, as well as extended culture over long periods of time with multiple cell passages.

Human Cells cultured in this way exhibit important responses that provide evidence of retained in vivo functional attributes as well as suitable signaling endpoints for other studies (gene expression, protein expression, morphology, excitation-contraction coupling, signaling mechanisms examined by phosphor-protein analysis etc). Very few changes in messenger RNA expression profiles were observed between myocytes cultured for 24 hours and freshly isolated cells. These results provide confidence that our model system is ready for additional studies and be explored to determine the complexity of the cardiomyocyte response to ligands, chemicals, and drugs. We have performed a broad screening of cardiomyocyte responses to approximately 130 ligands with measurements of changes in biochemical, anatomical, physiological, imaging-based, pathological as well as pathway responses, protein phosphorylation, contraction, and gene expression. These measurements will provide a spectrum of responses for comparison of individual ligands and for detection of interactions between combinations of ligands. Characterization of functional responses after extended times in culture is also now possible.

Primary Human Cardiomyocytes were obtained from dissociated heart tissue. Human adult heart tissues may also be available from vendors, for example, Celprogen Cat. No. 36044-15. A critical aspect in the culturing process was to bring the dissociated heart tissue to a single cell state and avoid the presence of cell clusters to ensure a successful and reproducible cardiomyocyte culture. After dissociation the cardiomyocytes are said to be in a P=0 state, which takes about a week to grow and become confluent in culture. As the cardiomyocyte passage advances in culture it takes 2-3 days to become confluent. It was important not to allow the cultured cardiomyocytes to reach overconfluence and splitting was done when cells reach approximately 90% confluence. During early passages, the splitting was done at ratios of between 1:5 and 1:10 (for example: to a 20 mL of media 1.5 to 2×10̂6 cells are added). For later passages, the splitting ratio was increased to between 1:20 and 1:25 as the cells are seeded at lower density. However, it was also crucial not to split the cells at densities that are too low since it will bring about clustering. Once the cardiomyocyte reached P=3, various tests (e.g. cardiotoxicity evaluation) could be performed and these tests could be performed until the cardiomyocytes reach P=12. Cells that have undergone more than twelve passages (>P12) were no longer used for tests.

Isolation and dissociation of heart tissue was done as follows. Tissue was transferred to ice cold calcium-free Krebs-Ringer saline solution and supplemented with taurine, carnitine, creatine, 2,3-butanedione monoxime, insulin, transferrin and selenium. Tissue dissociation utilized a combination of Krebs-Ringer solution supplemented with protease, Ringer saline containing a combination of collagenase A and hyaluronidase, collagenase A, and Krebs-Ringer solution with calcium.

Primary human cardiomyocytes in culture were grown in Medium 199 or DMEM with various combinations of Earle's salts, Hanks' salts, L-glutamine, sodium bicarbonate, and phenol red. The growth media was also supplemented with taurine, carnitine, creatine, 2,3-butanedione monoxime, insulin, transferrin, selenium, bovine serum albumin, with 2-10% bovine or fetal calf serum, penicillin, and streptomycin. During early passages, the media may also contain gentamicin and amphotericin.

Primary human cardiomyocytes have been grown and maintained in various culture formats including:
1. Glass cover slips
2. 6-well, 24-well, 48-well, 96-well and 384 well plates (black, white, opaque bottom, clear bottom)
3. 35, 60 and 100 mm tissue culture dishes
4. Various tissue culture flasks for ex: T75, T25
5. 1, 2, 4 and 8 glass chamber slides

After the cells have been seeded in any of the above culture plates, they were treated with test compound for periods generally ranging from 4 hours to 96 hours. Various assays were performed on these cells based on which mechanistic questions were being explored.

Characterization of Primary Human Cardiomyocyte Cells

For quality control we examined using RT-PCR more than 40 genes which play various roles in cardiac cells (for example: fatty acid metabolism, structure etc). Cells from 15 donors which ranged in passage numbers from P=0 to P=12 were used. The examined genes are listed in Table 1.

TABLE 1

| Symbol | Gene | Nucleotide ID |
|---|---|---|
| PDHA1 | Homo sapiens pyruvate dehydrogenase | NM_000284 |
| PFKFB1 | Homo sapiens 6-phosphofructo-2-kinase/fructose-2,6- | NM_002625. |
| | biphosphatase 1 | |
| PFKFB2 | Homo sapiens 6-phosphofructo-2-kinase/fructose-2,6- | NM_006212 |
| | biphosphatase 2 | |
| FABP3 | Homo sapiens fatty acid binding protein 3 | NM_004102 |
| CD36 | Homo sapiens CD36 molecule | NM_000072 |
| SLC27A1 | Homo sapiens solute carrier family 27 | NM_198580 |
| FASN | Homo sapiens fatty acid synthase | NM_004104 |
| CPT1A | Homo sapiens carnitine palmitoyltransferase I | NM_001876 |
| CPT2 | Homo sapiens carnitine palmitoyltransferase II | NM_000098 |
| ACACA | Homo sapiens acetyl-Coenzyme A carboxylase alpha | NM_198834 |
| ACOX1 | Homo sapiens acyl-Coenzyme A oxidase 1 | NM_004035 |
| ACADL | Homo sapiens acyl-Coenzyme A dehydrogenase, long chain | NM_001608 |
| | (ACADL | |
| ACADVL | Homo sapiens acyl-Coenzyme A dehydrogenase, very long | NM_000018 |
| | chain (ACADVL | |
| PRKAA1 | Homo sapiens protein kinase, AMP-activated, alpha 1 | NM_006251 |
| | catalytic subunit (PRKAA1 | |
| PRKAB1 | Homo sapiens protein kinase, AMP-activated, beta 1 non- | NM_006253 |
| | catalytic subunit (PRKAB1 | |
| ACTN1 | Homo sapiens actinin, alpha 1 | NM_001102 |
| ACTN2 | Homo sapiens actinin, alpha 2 | NM_001103 |
| NPPA | Homo sapiens natriuretic peptide precursor A | NM_006172 |
| NPPB | Homo sapiens natriuretic peptide precursor B | NM_002521 |
| BNP | Human brain natriuretic protein (BNP) gene | M31776 |
| TNNI3 | Homo sapiens troponin I type 3 (cardiac) (TNNI3 | NM_000363 |
| TNNC1 | Homo sapiens troponin C type 1 (slow) (TNNC1 | NM_003280 |
| TNNT2 | Homo sapiens troponin T type 2 (cardiac) (TNNT2), | NM_000364 |
| | transcript variant 1 | |
| CAV2 | Homo sapiens caveolin 2 | NM_001233 |
| CAV3 | Homo sapiens caveolin 3 | NM_001234 |
| NPPC | Homo sapiens natriuretic peptide precursor C (NPPC | NM_024409 |
| GJC1 | Homo sapiens Connexin-43 | NM_005497 |
| GATA4 | Homo sapiens GATA binding protein 4 | NM_002052 |
| FABP3 | Homo sapiens fatty acid binding protein 3, muscle and | NM_004102 |
| | heart | |
| IGF1 | Homo sapiens insulin-like growth factor 1 | NM_000618 |
| MYH7 | Homo sapiens myosin, heavy chain 7, cardiac muscle, beta | NM_000257 |
| MYH6 | Homo sapiens myosin, heavy chain 6, cardiac muscle, alpha | NM_002471 |
| | (cardiomyopathy, hypertrophic 1 | |
| MYL2 | Homo sapiens myosin, light chain 2, regulatory, cardiac, | NM_000432 |
| | slow (MYL2), MYL2v | |
| MYL7 | Homo sapiens myosin, light chain 7, regulatory (MYL7) | NM_021223 |
| | (MYL2a) | |
| NKX2-5 | Homo sapiens NK2 transcription factor related, locus 5 | NM_004387 |
| KCND3 | Homo sapiens potassium voltage-gated channel, Shal- | NM_004980 |
| | related subfamily, member 3 (KCND3) (kv4.3) | |
| CACNA1C | Homo sapiens calcium channel, voltage-dependent, N type, | NM_000719 |
| | alpha 1B subunit (CACNA1C) (CaV1.2) | |
| ATP2A2 | Homo sapiens ATPase, Ca++ transporting, cardiac muscle, | NM_001681 |
| | slow twitch 2 (ATP2A2), transcript variant 2, Serca2a | |
| PNMT | Homo sapiens phenylethanolamine N-methyltransferase | NM_002686 |
| VDAC1 | Homo sapiens voltage-dependent anion channel 1 (VDAC1) | NM_003374 |
| | (Porin) | |
| GPC3 | Homo sapiens glypican 3 | NM_004484 |
| GOT1 | Homo sapiens glutamic-oxaloacetic transaminase 1, soluble | NM_002079 |
| | (aspartate aminotransferase 1) | |
| KCNH2 | Homo sapiens potassium voltage-gated channel, subfamily | NM_000238 |
| | H (eag-related), member 2 (KCNH2), transcript variant 1, | |
| | mRNA | |
| ACTC | Cardiac actin | NM_005159 |
| DES | desmin | NM_001927 |
| FHL2 | four and a half LIM domains 2 | NM_001450 |
| MYBPC3 | Cardiac myosin binding protein C | NM_000256 |
| MYL3 | myosin essential light chain, cardiac | NM_000258 |
| PLN | phospholamban | NM_002667 |
| TPM1 | Tropomyosin 1 (alpha) | NM_000366 |

### Evaluation of Cardiac Phenotype

The following set of biomarker genes have been tested. We have done Immunofluorescence assay on Primary Human Cardiomyocytes for assay development. The list of antibodies examined so far include: Actinin, ANP, cKit, Connexin43, Desmin, KDR, NKX2.5, SERC2, SSEA1, SSEA3. SSEA4, TRA1-60, TRA1-81, Troponin I and Tropomycin.

Toxicity/Biomarker evaluation related to phospho-protein levels We have designed an assay procedure for measuring the phosphorylation of various endogenous proteins in Primary Human Cardiomyocytes. This high throughput assay is homogeneous and eliminates the need for performing western blotting. Phospho-proteins that have been tested (with phosphorylated amino acid residues) include: Phospho-4EBP 1 (Thr37/Thr46), Phospho-GSK 3a (Ser21), Phospho-JNK, Phospho-AKT (Ser473), Phospho-AKT (Thr308), Phospho-BAD (Ser112), Phospho-Caspase 9 (Ser196), Phospho-ERK 1/2, Phospho-GSK 3b (Ser9), Phospho-IGF-1 Receptor (Tyr1135/1136), Phospho-IkB (Ser32/Ser36), Phospho-IKKalpha (Ser176/Ser180), Phospho-mTOR (Ser2448), Phospho-mTOR (Ser2481), Phospho-IKKbeta (Ser177/Ser181), Phospho-Insulin Receptor (Tyr1150/1151), Phospho-NFkB p65 (Ser536), Phospho-p38 MAPK, Phospho-p70 S6K (Thr389), Phospho-S6 RP (Ser235/Ser236), Phospho-S6 RP (Ser240/Ser244), Phospho-STAT 3 (Tyr705), Phospho-ALK (Tyr1586), Phospho-ALK (Tyr1604), Phospho-Chk-1 (Ser345), Phospho-c-Jun (Ser 73), Phospho-c-Jun (Ser63), Phospho-EGF Receptor (Tyr1068), Phospho-ELK-1 (Ser383), Phospho-ErbB2 (Tyr1221/1222), Phospho-MEK 1, Phospho-PDK 1 (Ser241), Total ERK.

Toxicity Evaluation Through High-Content Imaging

We have optimized Primary Human Cardiomyocytes for use in high content imaging assays based on the following cellular features;

### Cellular Viability

Viability: Cell Tracker/Calcein (Molecular Probes)
Proliferation: BrdU, EdU and Ki67 Proliferation Markers (Molecular Probes)
Cytotoxicity: Membrane Permeability: Cellomics HCS; (Molecular Probes)

### Cellular Cytoskeletal Changes:

Actin (Phalloidin) combined with Tubulin (Anti-alpha Tubulin) plus DAPI (Molecular Probes)
Troponin I plus Troponin T combined with Hoechst 34222
Desmin plus Myosin Light Chain v2 with Hoechst 34222

### Organelle Health:

Peroxisomal Proliferation plus Lysosomal Content (PMP70+ Lysotracker+Hoechst 34222) (Molecular Probes)
Mitochondrial Function:
   Mitotracker Dye: Mitochondrial Membrane Potential, Mitochondrial Content in Cell (Molecular Probes)
   Mitochondrial Oxidative Phosphorylation Complex Evaluation HCS Evaluation—Complex I, II, III, IV, V Antibodies—Mitosciences, Molecular Probes
   Cytochrome C Release: Apoptosis (Molecular Probes)
   Porin Channel Function: (VDAC) Antibody based detection (Molecular Probes)
Stress
   ROS: Aminophenyl Fluorescein (APF) Dye Detection (Molecular Probes)(OCl, HO, ONOO)
   Oxidative Activity with Dichlorodihydroflurescein Diacetate CM-H2DCFDA-specific for cardiomyocytes
   Oxidative Stress plus Growth Factor Signaling: HIF-1, p-CREB, FOXO3a (Cellomics)
Cell Cycle
   P-Histone-H3 Antibody (HCS)
   CDK Cyclin D1, D2 (HCS)
   Rb Phosphorylation
   P53 activation (DNA Damage response) (Cellomics)
Apoptosis
   Necrosis/Permeability—YO-PRO, PI & Hoechst 34222 (Molecular Probes)
   DNA Strand Breaks-BrdU, EdU, PI Hoechst (Molecular Probes)'
   Cytochrome C relsease (HCS) (Molecular Probes)
   AIF & Bax Antibody based detection (Mitosciences) (HCS)
Energy Metabolism
   Lipid Accumulation: Phospholipid, Neutral Lipid (HCS) (Molecular Probes)

Biomarker Evaluation Through High-Content Imaging:

The following Protein Biomarkers are useful for tracking the onset of cardiotoxicity:

AKT, Phospho-AKT, Insulin Receptor B, IRS-1, IGF I Receptor B, Phospho IGF-I Receptor B, P27, AMPK alpha, Phospho-AMPK alpha, GSK-3B, Phospho-GSK-3B, Cytochrome c, mTOR, Phospho-mTOR

### EXAMPLES

### Cardiotoxicity Evaluation Through Biochemical Assays

A number of in vitro biochemical assays in which primary human cardiomyocytes were treated with test compounds evaluated for cardiotoxicity are described in the concurrently filed U.S. Provisional Patent Application by Bitter et al. entitled, “Assays To Predict Cardiotoxicity”, USSN 61/______, filed on Feb. 23, 2009 (Attorney Docket No. R0474A-PRO), which is incorporated herein by reference in its entirety.

Cardiomyocyte Attachment in Custom Made Plates

Cardiomyocytes were dispensed onto custom made pre-coated and uncoated plates. The plates were coated with a range of attachment factors including 10% FBS, 0.1% gelatin, matrigel, Fibronectin, collagen I and IV, laminin all reagents were of the purest quality obtained from BD Biosciences. Cardiomyocytes were dispensed directly onto substrates and cultured for 2-4 hours. Cell attachment was assessed by gently moving the culture dish and inspecting adherent cells by phase contrast light microscopy. Cells were cultured overnight and were scored on the basis of the number of cells remaining after medium was exchanged. The efficiencies of the attachment factors are summarized in Table 2.

TABLE 2

| Attachment factors | Atrial | Ventricular |
|---|---|---|
| Foetal bovine serum (10%) | + | + |
| 0.1% gelatin | + | + |
| Fibronectin | + | + |
| collagen I | ++++ | ++++ |
| collagen IV | + | + |
| Laminin | ++ | ++ |
| Laminin + Fibronectin | +++ | +++ |

Procedure for Cryogenic Preservation of Cells

Culture selection and examination: Prior to freezing, the culture should be maintained in active, growing state (log phase or exponential growth) to ensure optimum health and good recovery.
1. Using sterile pipette, aspirate the media completely.
2. Rinse the cell monolayer with 10 mL of sterile 1×PBS solution to remove any traces of serum.
3. Add 3 mL of 0.05% trypsin solution to T75 flask and incubate at 37° C. after gentle agitation for 5 min.
4. Examine the flask under the microscope to see if cells have started detaching. Once the cells have rounded up, gentle tapping of flask should detach the cells completely.
5. Add 10 mL of media to inactivate the trypsin to the flask. Perform vigorous pipetting 3-4 times to break of any cell clumps.
6. Collect the media in a sterile 15 mL centrifuge tube and centrifuge at 100 g for 5 minutes.
7. Transfer the centrifuge tube to the hood and aspirate the media without disturbing the pellet and resuspend the pellet in enough of the cell freezing medium to give a final cell concentration of 2-5 million viable cells/mL.
8. The cryoprotective agent contains 90% serum and 5% DMSO and 5% tissue culture growth medium.
9. Label the appropriate lot number, date, name of the cell and passage number on the vial. Add 1 to 1.8 ml of cell suspension to each of the vials and seal.
10. A slow and reproducible cooling rate is very important to ensure good recovery of the cultures.
11. Store cell cultures in −80 C.

Antibody staining in Primary Human Cardiomyocytes for Assay Development

Primary Human Cardiomyocytes were seeded (15000 cells/1.5 ml) in glass chamber for 24 hours at 37° C. Next day some of the chambers were treated with or without test compound and incubated for another 24 hours at 37° C. The following day cells were removed and washed with 1×PBS. Cells were fixed with 4.0% paraformaldehyde in PBS with for 30 minutes to 1 hour at room temperature and then washed with TBST0.1% (vol/vol) Tween-20 (TBST). Cell monolayer were blocked for at least 1 hour with a solution containing 0.1% (vol/vol) Tween-20 TBST+5% goat serum or 3% (wt/Vol) BSA or donkey serum. Following 1 hour incubation the slides were washed once with PBS or TBST. Next, primary antibody diluted with 5% goat serum-TBST was applied to the coverslip. The slides were incubated in a shaker for 1 hour at room temperature and then stored overnight at 4° C. The next day the slides were washed twice with TBST then secondary antibody diluted in TBST were added and allowed the incubate for one hour. The slides were then washed with TBST 2-3 times and DRAQ5 1:1000 was added followed by incubation for 15-30 minutes. Antibody staining was assessed with phase contrast light microscope.

Protocol for Measuring the Endogenous Phosphorlyation in Proteins:

Plate cells at a density of 20,000 cells per/100 μl in 96 well plates and incubate overnight at 37° C. The next day, cells were treated with compound to be tested along with reference compounds serving as positive and negative controls. The plates were incubated at 37° C. for 24 hours. The next day the media was removed and 1× lysis buffer (25 μl for 96 well plates) was added. The plates were gently shaken at 350 rpm for 15 minutes, after which 4 μl of the lysate was removed and placed 384 well plates. For a one-step assay, 7 μl of reaction buffer plus activation buffer containing alpha screen beads was added. For a two-step assay, 5 μl of reaction buffer plus activation buffer containing alpha screen acceptor beads was added. The plate was wrapped with foil and incubated at room temperature for either two hours or four hours with shaking at 350 rpm. In the one step procedure, the plate was read in an AlphaScreen-compatible reader after 4 hours or it can be stored at 4° C. for weeks in the dark for reading a later time. In the two step procedure, after 2 hours of incubation, 2.5 μl of dilution buffer containing alpha screen donor beads was added and the plate was shaken at 350 rpm for two additional hours. The plate was either immediately read in an AlphaScreen-compatible reader or stored at 4° C. for reading at a later time.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

What is claimed:

1. A method of screening an agent for a modulatory effect on primary human cardiomyocytes comprising:
a) culturing said primary human cardiomyocytes,
b) plating said primary human cardiomyocytes on multi-well plates selected from the group consisting of 6-well plates, 24-well plates, 48-well plates, 96-well plates, and 384-well plates.
c) contacting said primary human cardiomyoctyes with the agent; and
d) examining said primary human cardiomyocytes for the modulatory effect resulting from said agent.

2. The method of claim 1 wherein said modulatory effect on primary human cardiomyocytes is selected from the group consisting of cardiotoxicity, differentiation, proliferation, survival, change in metabolic activity, change in biochemical activity, and change in contractile activity.

3. The method of claim 2 wherein said modulatory effect on primary human cardiomyocytes is cardiotoxicity.

4. The method of claim 3 wherein said cardiotoxicity on primary human cardiomyocytes is determined by performing at least one experiment selected from the group consisting of:
biochemical assays, biomarker evaluation related to phosphor-protein levels, toxicity evaluation through high-content imaging, and biomarker evaluation through high-content imaging.

5. The method of claim 4 wherein said experiment is a biochemical assay.

6. A method of screening an agent for a modulatory effect on primary human cardiomyocytes comprising:
a) culturing primary human cardiomyocytes for at least three passages (P=3) in growth medium comprising of Medium 199 or DMEM, 2-10% bovine or fetal calf serum, on plates coated with collagen I;
b) plating said primary human cardiomyocytes on multi-well plates selected from the group consisting of 6-well plates, 24-well plates, 48-well plates, 96-well plates, and 384-well plates;
c) contacting said primary human cardiomyoctyes with the agent; and
d) examining said primary human cardiomyocytes for the modulatory effect resulting from said agent.

7. The method of claim 6 wherein said modulatory effect on primary human cardiomyocytes is selected from the group consisting of cardiotoxicity, differentiation, proliferation, survival, change in metabolic activity, change in biochemical activity, and change in contractile activity.

8. The method of claim 7 wherein said modulatory effect on primary human cardiomyocytes is cardiotoxicity.

9. The method of claim 8 wherein said cardiotoxicity on primary human cardiomyocytes is determined by performing at least one experiment selected from the group consisting of:
biochemical assays, biomarker evaluation related to phosphor-protein levels, toxicity evaluation through high-content imaging, and biomarker evaluation through high-content imaging.

10. The method of claim 9 wherein said experiment is a biochemical assay.
